(19) United States
(12) Patent Application Publication
(10) Pub. No.: US 2025/0228688 A1
(43) Pub. Date: Jul. 17, 2025

(54) TUNING KIT AND RELATED METHOD FOR TUNING ANKLE FOOT ORTHOSIS

(71) Applicant: Launchpad O & P LLC, Minneapolis, MN (US)

(72) Inventor: Patrick Scott Hinshon, Maplewood, MN (US)

(21) Appl. No.: 18/414,420

(22) Filed: Jan. 16, 2024

Related U.S. Application Data
(60) Provisional application No. 63/479,893, filed on Jan. 13, 2023.

(51) Int. Cl. A61F 5/01

(52) U.S. Cl. CPC A61F 5/0111

(57) ABSTRACT

A tuning kit including multiple tuning components provides an orthotists with the ability to custom tune an Ankle-Foot-Orthosis (AFO) without requiring permanent adjustments. This kit will further allow for the continued readjustment of AFOs as therapy progresses. Tuning components will include heel lifts (boosters), toe extensions, intrinsic forefoot posts (both medial and lateral) and extrinsic full-length wedges (both medial and lateral). A related tuning method allows for the efficient and effective turning of AFOs.

### BACKGROUND

Many different devices have been utilized in the past to assist with lower extremity conditions (diagnoses) that lead to compromised alignments, contracture, and weakness that cause gait abnormalities. One device typically used for these situations is an ankle foot orthosis (AFO), which helps provide support and/to assist during both stance and swing phase throughout the gait cycle. AFOs further help patients manage or establish a more stable and/or normalized walking gait, by providing support and/or stop motion and/or resistance as needed to portion of the user's foot, ankle and knee. AFOs come in many different forms and often provide specific functions and/or support, based on the functional and rehabilitative goals of the clinicians and patients. More specifically this could include restoring range of motion, improving mobility, balance, endurance, distance or pain/deformity.

Various details of certain AFOs can be found in U.S. Pat. No. 11,278,439 entitled “Ankle-Foot Orthosis and issued to the named inventor of the present application. Additional options are discussed in US Published Patent Application Publication 2020/0383815, also filed by the named inventor of the present application. As discussed in this Publication, control and customization options for practitioners is beneficial. The AFO described therein is articulated, includes plantar flexion stops to appropriately limit plantar flexion of the user's foot, while also providing dorsi-flexion resistance and/or stops to control dorsi-flexion. In the various embodiments, a dorsi-flexion control member 100 or 100′ is added.

As shown, FIG. 1A illustrates an example AFO 110*, which is articulated about a hinge point 55. A joint 50 is provided on each side of AFO 110* to accommodate articulation of an upper boot member 20 and a lower boot member 30. Further, AFO 110* includes a stop 160 on the back side to provide plantar-flexion stops, as desired. Many different stops 160 could be used, including for example the stop device set forth and described in U.S. Pat. No. 7,018,350, issued in 2006 to the named inventor of the present application. Turning now to FIG. 1B, the illustrated AFO 110 again has an articulated upper boot member 120 and a lower boot member 130, which are coupled together by joint 150. Here, AFO 110 includes a dorsi-flexion control member 100, to help control the dorsi-flexion of the user's ankle joint. In FIG. 1C AFO 110′ has an articulated upper boot member 120 and a lower boot member 130. To provide an alternative approach however, a static dorsi-flexion control member 100′ is attached at a rear location of AFO 110′.

As is well known, traditional AFOs typically combine multiple materials to achieve the necessary support functions, while also providing comfort and usability. That said, the selection configuration and characteristics of the various components involved greatly affect the way an AFO operates, and the related treatment/therapy provided to a patient.

The ability to adjust AFO alignment in the sagittal, coronal, and transverse planes through tuning is necessary to achieve optimal static and dynamic function. During a fitting appointment, the device is fit to a properly sized shoe and tuned to the optimal (normal) shank to vertical alignment and foot progression angles (intoeing or out-toeing) by adjusting the alignment of the orthosis with tuning devices.

When a lower limb orthosis is delivered to a patient it goes through 3 phases of alignment (bench, static, & dynamic). These stages of alignment are to normalize gate and enable patients to accomplish their goals, ADLs, vocational requirements, and exercise activities. The alignable system can be in the form of componentry that allows the orthotist to optimize the patient's gait. To accomplish gait optimization the orthotist uses materials (e.g., rigid foam, plastic, leather) in combination with the orthosis and work in conjunction with the shoe to properly align the AFO/KAFO in all three planes. This process requires observational gait assessment as well as using other tools (e.g., gait mat, video, gait lab, etc.) to validate and measure the alignment and outcomes.

### SUMMARY

As further discussed in detail below, a method of alignment, adjustment and tuning described herein provides additional efficiency and benefits when treating patients. Further, this process includes the use of a tuning kit that is specifically designed to provide an orthotist with, posts, wedges, pads that are place intrinsically (inside) and extrinsically (underneath) the AFO to provide optimal gait mechanics and/or therapeutic benefits for the patient. One example tuning kit may include heel lifts, toe extensions, various medial and lateral forefoot posts, full length wedges, and full-length pads. Further, each of these components can be provided in various thicknesses and various levels of hardness, thus providing the tools necessary for appropriate tuning of a prescribed AFO. It is noted that this tuning kit and the related methods are valuable during an initial fitting and during continued therapy. It is additionally noted that the process outlined below can be adapted to provide tuning for both articulated AFOs and non-articulated AFOs.

### BRIEF DESCRIPTION OF THE DRAWINGS

Additional details of the disclosed embodiments are set forth in description below in conjunction with the drawing figures, in which:

FIGS. 1A-1C show three example AFOs;

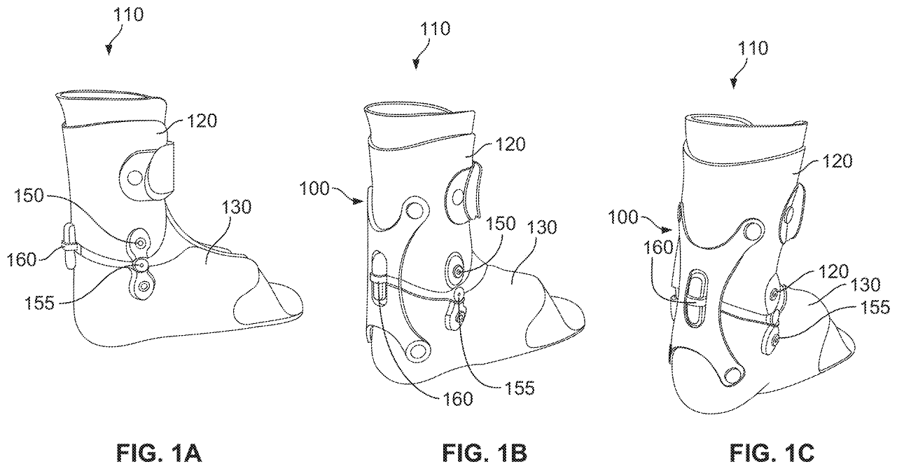

FIGS. 2A & 2B illustrate the process of measuring a shank to vertical angle (SVA);

FIG. 3 presents a view of an individual in a terminal stance position during a normal stride;

FIG. 4, including FIGS. 4A-4C, illustrates a heel lift;

FIG. 5 graphically illustrates the intended position of a heel lift;

FIG. 6 shows a toe extension;

FIG. 7 presents a medial forefoot post;

FIG. 8 Illustrates a lateral forefoot post;

FIG. 9 presents a lateral full-length wedge; and

FIG. 10 shows a medial full-length wedge.

### DESCRIPTION

As discussed below, a method of alignment, adjustment and tuning described herein provides additional efficiency and benefits when treating patients. Further, this process includes the use of a tuning kit that is specifically designed to provide an orthotist with tools to tune the AFO in a manner which will provide optimal support and/or therapy for the patient. One example tuning kit may include heel lifts, toe extensions, various forefoot posts and full-length wedges. Further, each of these components can be provided in various thicknesses and hardness, thus providing the tools necessary for appropriate tuning of a prescribed AFO. It is additionally noted that the process outlined below can be adapted to provide tuning for both articulated AFOs and non-articulated AFOs. Additionally, while the discussion below summarizes the use of various tools that are often applied to inner portions of an AFO, it is understood that situations exist where tools are applied to the outer portion of the AFO or to portions of the patient's shoes.

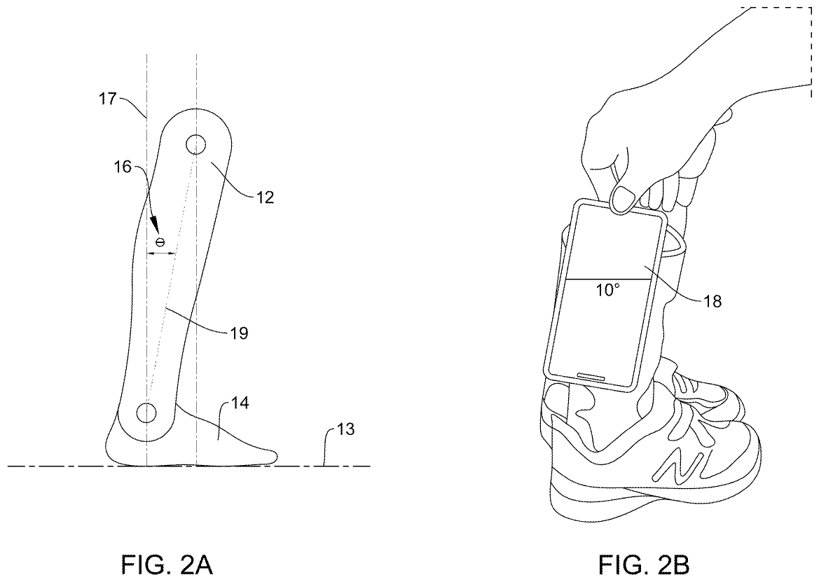

In the tuning process, an initial goal involves analyzing and tuning the Shank-to-Vertical Angle (SVA). As the name indicates, this is a measurement of the ankle/shin's alignment with vertical as it exists in the sagittal plane. The details of the SVA patent's measurement are generally illustrated in FIGS. 2A & 2B, wherein FIG. 2A shows schematic details of the SVA measurement, while FIG. 2B illustrates one approach to this measurement process using a mobile device 18. In FIG. 2A, a representation of a patient's lower leg 12 and foot 14 are presented, as would be arranged when the patient is standing on the floor 13. The SVA 16 is shown, which is the angle between a vertical axis 17 and a lower leg axis 19. The SVA measurement can be made when the AFO is being worn, or without the AFO and provides valuable information in each situation. As will be appreciated, the measured SVA is generally related to the diagnosis presented, (i.e., Spina Bifida, Cerebral Palsy, . . . ) and the treatment plan being followed. In practice, the AFO is typically set to a desired bench alignment depending on the type of AFO being used (e.g., solid ankle v. articulated ankle AFO).

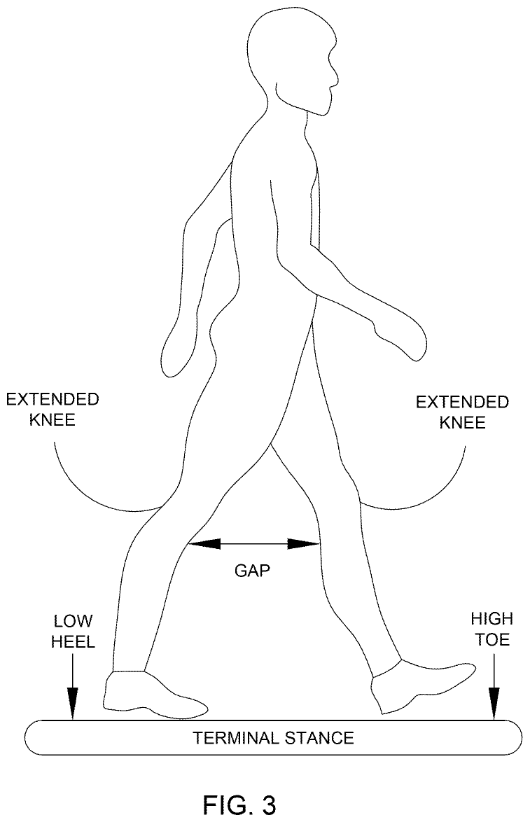

In an effort to provide the most optimal therapy plan for patients, the AFO tuning process begins by identifying the type of AFO involved which can be dependent upon many different factors, which orthotists will appreciate and consider. The type of AFO chosen will then dictate a target SVA. Next, videos should be taken of the patient walking to show views of both the Coronal plane and the Sagittal plane. The video will then be analyzed, looking primarily for dynamic alignment. More specifically, the process will first look at alignment in the sagittal plane, and an analysis of alignment is completed when the patient is in a predetermined position (i.e. a terminal stance position). As generally shown in FIG. 3, a patient is at this predetermined position when at the terminal stance phase of the gait. Stated differently, during a normal stride, the patient is in the predetermined position when: (a) the front leg is in a high toe position with the front knee extended; (b) the back leg has the back knee extended; (c) the heel of the back foot is low; and (d) the rear hip is extended. In addition, the patient's trunk is upright the position, perhaps with a slight lean forward (as is typical during a normal stride). Reviewing video or images of the patient in this position, an analysis of foot, leg and body positions is evaluated. Again, this is generally illustrated in FIG. 3, showing a patient in the terminal stance phase of the gait where the space between the patient's legs resembles an “A” or inverted “V”. The analysis and fitting process may include measurements of stride length, the length of the gap between the knees, and the angle of the legs with respect to the floor/ground. These measurements can then be used for AFO fitting/tuning.

A similar analysis of the coronal plane view will then be completed to develop additional data. Looking again at terminal stance positions, this analysis is looking for straight foot projections and a normal base of support. Further, measurements related to leg spacing and leg angles can provide valuable information. Again, this is generally reviewed using video and/or images of the patient during normal walking conditions.

The video analysis and measurements suggested above is looking to see if the patient's walking stride or gait is within a preferable range, and to determine if additional adjustments or tuning would be helpful. In those situations where video/image analysis reveals that further tuning or adjustments are necessary, the orthotist can them make use of other tools included in a tuning kit which may include some or all of the components or tuning tools discussed below. While variations are possible, the tuning kit may include a heel lift 20, a toe extension 30, a medial forefoot post 40, a lateral forefoot post 50, a lateral full-length wedge 60, and a medial full-length wedge 70. Each of these components are provided in differing sizes which are dimensioned to fit the patient's AFO, shoe or foot, with multiple thicknesses and multiple levels of firmness available. As will be discussed below, the multiple options provided in the tuning kit provides the ability to custom fit products for patients as part of their therapy.

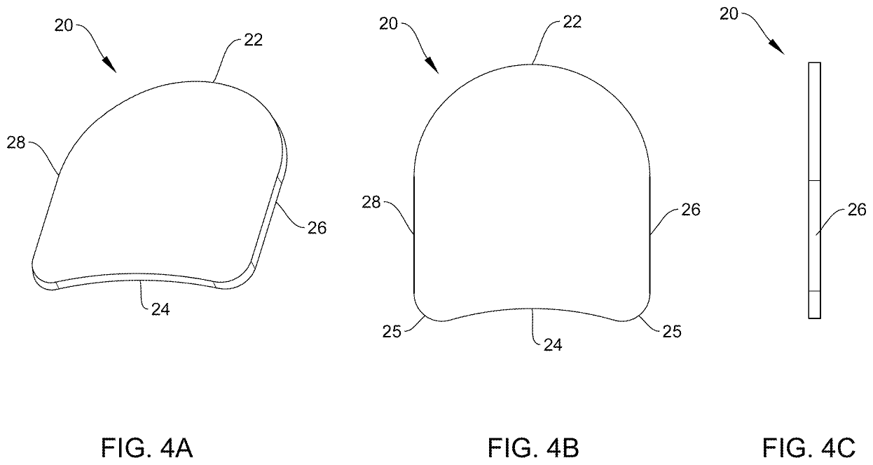

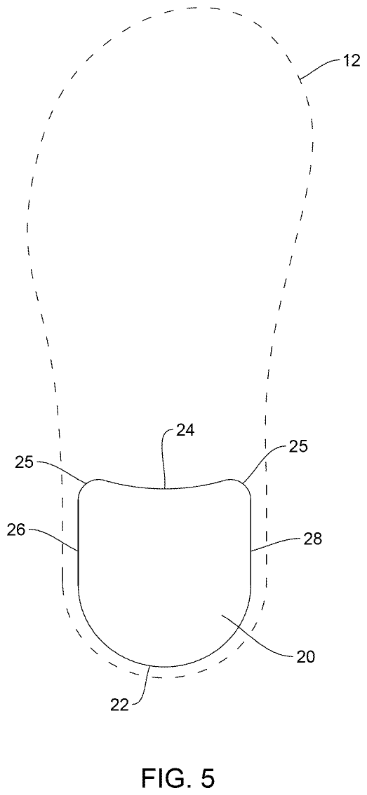

As an initial starting point, a first component of the tuning kit typically used to set an initial SVA is a heel lift 20. One example of heel lift 20 is illustrated in FIGS. 4A-4C, with a perspective view shown in FIG. 4A, a top view shown in FIG. 4B and a side view shown in FIG. 4C. It is noted that the specific shape and the thickness of heel lift 20 can vary depending on the specific application. In practice, an appropriately sized heel lift 20 is placed within the prescribed AFO to obtain a desired starting SVA. Alternatively, heel lift 20 could be applied to an outer portion of the AFO, or could be placed within the patient's shoe. Where necessary, it is contemplated that various adhesives would be used for attachment (including tape, glue or other liquid adhesives). Once this is established, further analysis is done to review knee, hip and trunk alignment. Again, one component in the above-mentioned tuning kit 10 is the heel lift 20 (i.e., booster), which is further illustrated in FIG. 5. More specifically, FIG. 5 further illustrates the relative placement of heel lift 20 in this embodiment, by showing an outline 12 of a patient's shoe insole (which generally tracks the outline of the patient's foot.) It has been found that heel lift 20 can be used in many situations and can help to achieve several “goals”. For example, heel lift 20 can help to increase SVA and thus improve tibial progression. Further, heel lift 20 can be used to straighten foot projections when SVA is insufficient. In addition, when used heel lift 20 will help to increase step length and/of cadence, while also helping to prevent knee hyperextension and early heel rise. As illustrated, heel lift 20 has a distal edge 22 which is curved and configured to match a patient's heel while also fitting within any shoe that will be used. In this embodiment, distal edge is semi-circular having a constant radius of curvature. Here, the corners 25 of proximal edge 24 are rounded or curved to again accommodate fitting. A first side 26 and a second side 28 are further configured to provide appropriate transitions and fit the contours of the related AFO. Again, various thicknesses can be used to fine-tune the patient's gait and can be exchanged as therapy progresses. It is generally contemplated that heel lift 20 will be available in thicknesses ranging from less than ¼ inches to over ½ inches, although other thicknesses may be necessary in other circumstances. Further, the firmness can be varied, with

[symbol]

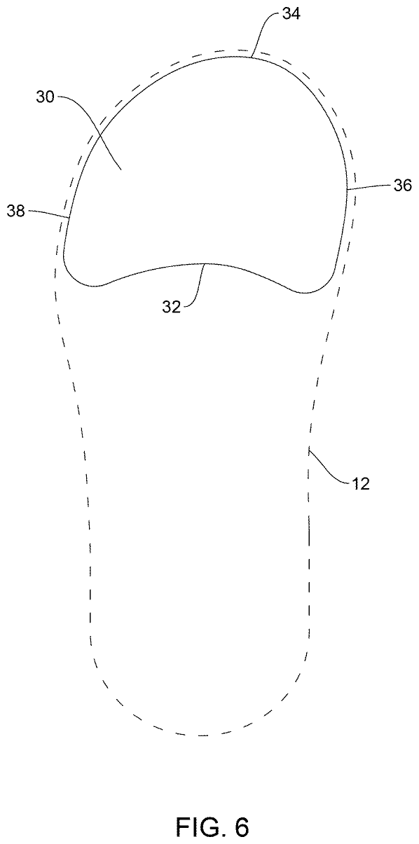

As also mentioned, the tuning kit will include toe extensions 30, as shown in FIG. 6. Although the name is somewhat suggestive of the desired positioning, FIG. 6 shows where toe extension 30 would typically be placed in an AFO and how it would be aligned with a patient's foot. Further, toe extension has a proximal edge 32, a distal edge 34 and sides 36, 38 all shaped and configured to fit within the related AFO and provide appropriate support and toe extension, for related portions of a patient's foot. When used, toe extensions 30 can help to encourage posterior weigh shift, but can also increase gastroc-soleus muscle length, decrease gastroc-soleus tone, prevent toe curling/gripping, and can assist with a patient's pre-swing push-off. It is generally anticipated that toe extension 30 could be provided in a ¼ inch high-durometer foam (e.g., crepe), a ½ inch high-durometer foam (typically used to create a greater posterior weight shift) or a ¼ inch medium-durometer foam (e.g., trilam). When used, the proximal edge 32 should be skived to provide a smooth transition for the toes.

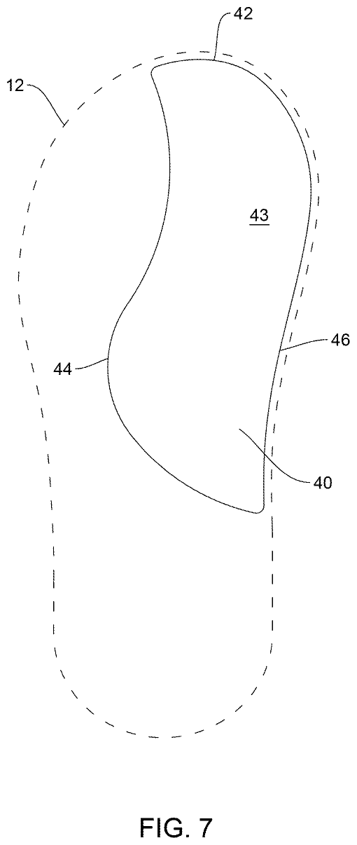

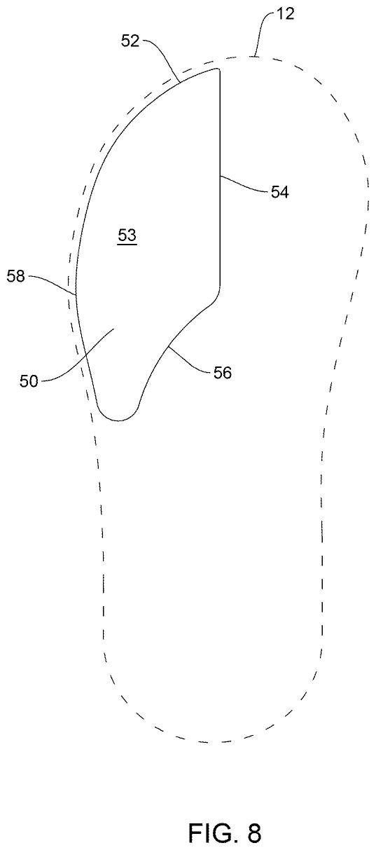

Intrinsic forefoot posts are also part of the tuning kit, including medial forefoot post 40 (see, FIG. 7) and lateral forefoot post 50 (see, FIG. 8). Turning now to medial forefoot post 40 shown in FIG. 7, this component can be used to deal with externally rotated foot projections and pronated foot posture. In one embodiment, medial forefoot post 40 has a distal end 42 which is adjacent to a forefoot portion 43 which is intended to be positioned under the patients' metatarsals and toes (i.e., under the front medial portion of a patient's foot). A proximal arch portion 44 is configured to be positioned beneath a patient's longitudinal arch, while a curved outer edge 46 is configured to be positioned beneath the medial edge of a patient's foot. Further, in this particular embodiment medial forefoot post 40 is formed of ¼ inch medium-durometer foam. Those skilled in the art will recognize that other variations may be appropriate for other situations.

FIG. 8 presents a view of lateral forefoot post 50, again overlaid upon an outline 12 of the patient's foot. Here lateral forefoot post can be used to address internally rotated foot projections and/or supinated foot posture. Lateral forefoot post 50 includes a distal edge 52 which is adjacent to a forefoot portion 53 which is intended to be positioned below the patient's lateral metatarsals and toes (i.e., under the front lateral portion of a patient's foot). A central edge 54 is configured to be positioned beneath the central portion of the patient's forefoot. A curved inner edge 56 is configured to generally sit adjacent the transition between the patient's forefoot and arch, while a curved outer edge 58 is configured to be positioned beneath the medial edge of a patient's foot. In this embodiment lateral forefoot post 50 will be formed of ¼ inch high-durometer foam (e.g., crepe) or ½ inch high durometer foam (typically used with patients having significantly supinated foot postures). Again, in application the edges of lateral forefoot post 50 should be skived to provide a smooth transition for the patient's toes.

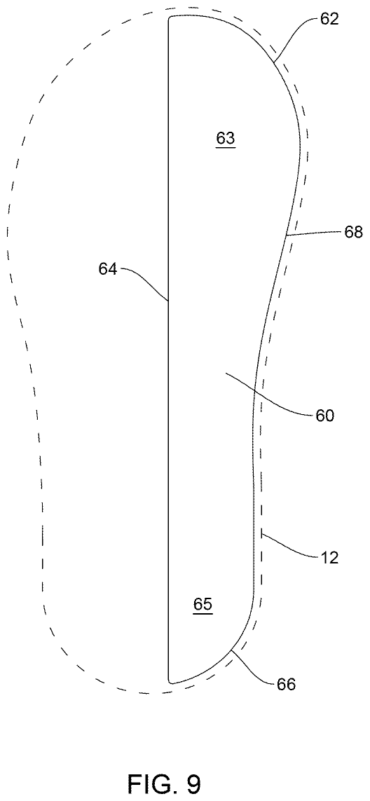

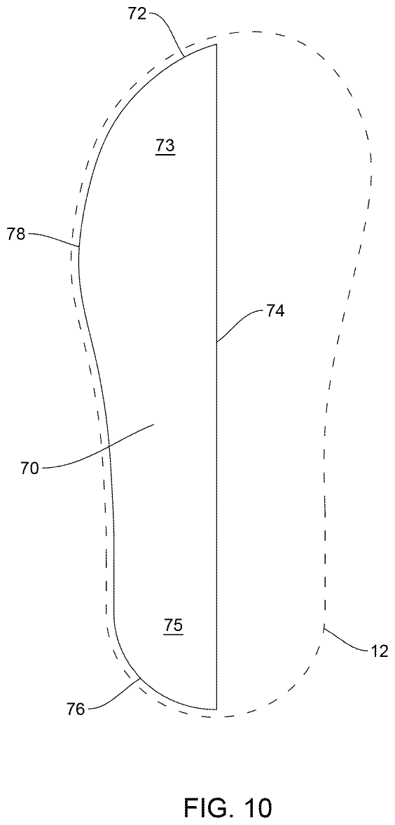

In addition to the tools mentioned above, extrinsic full-length wedges, including lateral full-length wedge 60 (see FIG. 9) and medial full-length wedge 70 (see FIG. 10) can be included as part of the tuning kit. Lateral full-length wedge 60 is often used to address internally rotated foot projections, and/or genu varum. As shown in FIG. 9, lateral full-length wedge 60 has a distal edge 62 adjacent to a forefoot portion 63, a central edge 64, a proximal end 66 and a lateral edge 68. Again, lateral edge 68 is intended to sit below the lateral edge of a patient's foot. A heel portion 65 is intended to sit below the patient's heel. Lateral full-length wedge 60 can be formed from a ¼ inch high-durometer foam, or a ½ inch high-durometer foam, as needed.

Referring now to FIG. 10, medial full-length wedge 70 is again shown overlaid upon the outline 12 of a patient's foot. As presented in FIG. 10 medial full-length wedge 70 has distal end 72 which is adjacent a forefoot portion 73, a proximal edge 76 adjacent a heel portion, a central edge 74 and a medial edge 78 which is configured to be below the medial edge of the patient's foot. Those skilled in the art will recognize that medial full-length wedge 70 can be used to address conditions of externally rotated foot projection and/or genu valgum. Medial full-length wedge 70 can also be formed from a ¼ inch high-durometer foam, or a ½ inch high-durometer foam, as needed. Other alternatives are possible.

Again, each of these tools allow for related adjustment of alignment and provides the ability to custom fit an AFO to a patient, including the custom fitting of an articulated AFO or a non-articulated AFO. By having a tuning kit that includes several components, the process of tuning an AFO can be easily completed by an orthotist without having to make permanent adjustments. This kit will thus increase efficiency and effectiveness of any prescribed AFO. Further, the kit can be packaged in many different ways, including portable containers or carriers. The kit could also be contained within an organizer that allows quick and easy access to each of the components, while also including tools necessary to make modifications (e.g., cutting and trimming devices, additional adhesive, etc.)

Various embodiments of the invention have been described above for purposes of illustrating the details thereof and to enable one of ordinary skill in the art to make and use the invention. The details and features of the disclosed embodiment[s] are not intended to be limiting, as many variations and modifications will be readily apparent to those of skill in the art. Accordingly, the scope of the present disclosure is intended to be interpreted broadly and to include all variations and modifications coming within the scope and spirit of the appended claims and their legal equivalents.

1. A method for tuning an AFO for a patient, comprising:
Evaluating a patient's condition and measuring a patient's Shank-to-Vertical Angle (SVA);
defining the desired AFO and a desired target range for the patient's SVA;
measuring the patient's SVA while wearing the AFO;
selecting at least one first tuning component, selected from the group of a heel lift, a toe extension, a medial forefoot post, a lateral forefoot post, a lateral full-length wedge, and a medial full-length wedge, setting up the AFO with the selected tuning component to achieve an SVA within the desired target range;
analyzing the use of the AFO by the patient by looking at a front leg position of the patient, a front knee position of the patient, a front toe position of the patient, a front foot position of the patient, a back leg position of the patient, a back knee position of the patient, and a rear heel position of the patient, all when the user is in a terminal stance position; and
further tuning the AFO using a second selected one of the tuning components.

2. The method of claim 1 wherein the kit further comprises a plurality of heel lifts with each having a different thickness, a plurality of heel locks, a plurality of intrinsic heel lifts, a plurality of toe extensions, a plurality of intrinsic forefoot posts, and a plurality of extrinsic full-length wedges.

3. The method of claim 1 wherein selecting the at least one tuning component further comprises selecting from a ¼ inch low durometer heel lift, a ½ inch low durometer heel lift, a ¼ inch high-durometer heel lift, a ½ inch high-durometer foam toe extension, a ¼ inch medium-durometer foam toe extension, a ¼ inch low durometer toe extension, a ¼ inch medium-durometer foam medial forefoot post, a ¼ inch medium-durometer foam lateral forefoot post, a ½ inch high durometer foam lateral forefoot post, a ¼ inch high-durometer foam lateral full-length wedge, a ½ inch high-durometer foam lateral full-length wedge, a ¼ inch high-durometer foam medial full-length wedge, and a ½ inch high-durometer foam medial full-length wedge.

4. The method of claim 3 wherein the at least one first tuning component is applied to an inner portion of the AFO.

5. The method of claim 4 wherein the second selected one of the tuning components of the tuning components is applied to an outer portion of the AFO.

6. The method of claim 5 wherein the at least at least one first tuning component is removed before the second selected one of the tuning components is utilized for tuning.

7. The method of claim 3 wherein the at least one first tuning component is applied to an outer portion of the AFO.

8. The method of claim 7 wherein the second selected one of the tuning components of the tuning components is applied to an inner portion of the AFO.

9. The method of claim 8 wherein the at least at least one first tuning component is removed before the second selected one of the tuning components is utilized for tuning.

10. The method of claim 3 wherein the at least one first tuning component is applied within a shoe of the patient.

11. The method of claim 10 wherein the second selected one of the tuning components of the tuning components is applied to an inner portion of the AFO.

12. The method of claim 1 wherein the at least one first tuning component is a heel lift having a predetermined thickness and predetermined hardness which is applied to an outer portion of the AFO.

13. A tuning kit for tuning the operating characteristics of an AFO, the tuning kit having a plurality of adjustment components, comprising:
a plurality of heel lifts;
a plurality of intrinsic heel lifts;
a plurality of toe extensions;
a plurality of intrinsic forefoot posts; and
a plurality of extrinsic full-length wedges.

14. The kit of claim 13 wherein the plurality of adjustment components are provided in differing thicknesses and differing hardnesses.

15. The kit of claim 13 wherein the plurality of heel lifts comprise a ¼ inch low durometer heel lift, a ½ inch low durometer heel lift, and a ¼ inch high-durometer heel lift.

16. The kit of claim 13 wherein the plurality of toe extensions comprise a ½ inch high-durometer foam toe extension, a ¼ inch medium-durometer foam toe extension, and a ¼ inch low durometer toe extension.

17. The kit of claim 13 wherein the plurality of forefoot posts comprise a ¼ inch medium-durometer foam medial forefoot post, a ¼ inch medium-durometer foam lateral forefoot post, and a ½ inch high durometer foam lateral forefoot post.

18. The kit of claim 13 wherein the plurality of full-length wedges comprise a ¼ inch high-durometer foam lateral full-length wedge, a ½ inch high-durometer foam lateral full-length wedge, a ¼ inch high-durometer foam medial full-length wedge, and a ½ inch high-durometer foam medial full-length wedge.
